(19) United States
(12) Patent Application Publication
(10) Pub. No.: US 2018/0049952 A1
(43) Pub. Date: Feb. 22, 2018

(54) METHOD FOR PRODUCING DENTAL PROSTHESES, READY-TO-USE DENTAL MATERIAL, AND KIT CONTAINING THE DENTAL MATERIAL

(71) Applicant: KULZER GMBH, Hanau (DE)

(72) Inventors: Klaus RUPPERT, Maintal (DE); Alfred HOHMANN, Schmitten (DE); Stephan DEKERT, Wehrheim (DE)

(73) Assignee: KULZER GMBH, Hanau (DE)

(21) Appl. No.: 15/560,631

(22) PCT Filed: Mar. 22, 2016
(86) PCT No.: PCT/EP2016/056261

§ 371 Date: Sep. 22, 2017

(30) Foreign Application Priority Data
Mar. 24, 2015 (DE) 10 2015 104 440.4

(51) Int. Cl. A61K 6/00; A61K 6/083

(52) U.S. Cl. CPC A61K 6/0002; C08L 33/04; A61K 6/083

(57) ABSTRACT

A method for the production of dental prosthetic moulded parts as well as a ready for use, polymerisable dental material for the production of dental prosthetic moulded parts, such as of total or partial prostheses, parts of bridges, crowns, is proposed. The ready for use dental material, optionally after mixing the separated two paste dental material having an initiator system separated into the pastes and otherwise being essentially identical dental material, is immediately curable or polymerisable, respectively, into prosthetic moulded parts without swelling time. A kit comprising the ready for use, polymerisable dental material, as well as the use of the kit and a method for the production of dental prosthetic moulded parts is also a subject matter of the invention.

A method for the production of dental prosthetic moulded parts as well as a ready for use, polymerisable dental material for the production of dental prosthetic moulded parts, such as of total or partial prostheses, parts of bridges, crowns, is proposed. The ready for use dental material is preferably used in the method according to the invention. The ready for use dental material, optionally after mixing the separated two paste dental material having an initiator system separated into the pastes and otherwise being essentially identical dental material, is immediately curable or polymerisable, respectively, into prosthetic moulded parts without swelling time. A kit comprising the ready for use, polymerisable dental material, as well as the use of the kit and a method for the production of dental prosthetic moulded parts is also a subject matter of the invention. The dental material comprises an initiator or an initiator system for redox reaction (cold-curing) and heat-curing (hot-polymerisation).

Prosthesis materials on (meth)acrylate basis usually consist of a powder-liquid system resulting, by mixing of the two components in a defined ratio, in a processable dough after waiting time and swelling time. Said dough is then casted, pressed or injected into the cavity, mostly a cuvette comprising a negative mould of the prosthesis to be produced, for the prosthesis to be produced and the polymerisation is subsequently initiated by redox reaction in the case of cold-polymerisates (e.g. amine-peroxide or barbituric acid), by thermal decay, e.g. of a peroxide, in the case of hot-polymerisation.

An essential disadvantage of the known method and the known compositions is that the two usual main components of the dental materials, PMMA powder and MMA liquid, are mixed by intensive stirring. Said mixing normally takes approx. 30 sec. The mixing is then followed by a swelling time, in which the mixture swells to a processable, pasty dough (also referred to as “putty” or “dough”). Said swelling time after mixing usually takes approx. 10 to 15 minutes in the case of hot-polymerisates and approx. 1 to 3 minutes in the case of cold-polymerisates. Aside considerable time requirement, at this there is always a risk of introducing air bubbles into the material by stirring, thereby reducing the mechanical strength of the prosthesis.

A further potential source of error is non-compliance with the mixing ratio by the user, which may also adversely affect the quality of the prosthesis material. On the one hand the mechanical strength may decrease, on the other hand increased amount of residual monomer may occur including health risk relating thereto for the patient.

In addition, a certain waiting time must be complied after mixing powder and liquid in order to allow dissolving and swelling of methylmethacrylate and PMMA beads. A processable dough only arises by this swelling process. Said interruption of the workflow is disruptive and undesired in dental lab.

The consistency of the dough is typically reviewed by hand by the user since the swelling process also depends on ambient temperature, hereby skin allergies and incompatibilities may occur or be enhanced, respectively.

In addition, considerable exposure with MMA vapours as well as dust particles from PMMA powder typically occur by open mixing of the powder component with the liquid component in the dental lab.

An object of the invention was to indicate a method and a dental material for the production of dental prostheses, in which the disadvantages, described above, in the production of prosthesis materials are omitted or minimized. Thus, an object was, the development of a method reliably avoiding exposure and contamination with monomer vapours and dust particles in the production of dental prosthetic moulded parts and preferably reducing the processing time at the same time. Furthermore, the object was to indicate a dental material, preferably being free of MMA, and essentially polymerises completely, thus not having any residual monomer content anymore after polymerisation. In addition, a dental material was to be provided which may be used without swelling time and no qualitative impairment concerning mechanical strength such as, for example, flexural strength, E-modulus or fracture toughness occur. In addition, the dental material must further be storable.

According to the invention, the object is solved by the method according to the invention according to claim 1. Furthermore, the object is solved by the dental material according to the invention according to claim 5. Further preferred embodiments are disclosed in the subclaims as well as in the description.

Subject matter of the invention is a method for the production of dental prosthetic moulded parts, in particular of a dental total or partial prosthesis, comprising the steps of:
using, preferably providing, a ready for use, polymerisable dental material as at least one paste, in particular having a viscosity of 1000 mPa·s to 75000 mPa·s, in particular of 1000 mPa·s to 50000 mPa·s, preferably of 1000 mPa·s to 20000 mPa·s, particularly preferably of 2000 mPa·s to 15000 mPa·s, in at least one or two cartridge(s),
discharging the ready for use dental material, optionally my mixing the dental material, and
charging the dental material into a negative mould for the production of at least one dental prosthetic moulded part, and
optionally, polymerising the dental material and, preferably obtaining a dental prosthetic moulded part, in particular a dental total- or partial prosthesis having a flexural strength of greater than or equal to 60 [MPa], preferably greater than or equal to 62 [MPa], further preferably greater than or equal to 64 [MPa], in particular up to 100 [MPa], and/or having an E-modulus of greater than or equal to 1500 [MPa], in particular greater than or equal to 1900 [MPa], preferably greater than or equal to 2100 [MPa], further preferably greater than or equal to 2200 [MPa], in particular up to 3500 [MPa]. The afore-mentioned parameters for flexural strength and E-modulus are determined in particular according to DIN ISO 20795-1: 2013-06.

According to the invention, the ready for use, polymerisable dental material is not a light-curing polymerisable dental material, but a heat-curing dental material or an autopolymerising dental material, in particular an autopolymerising 2K-dental material and, preferably, the dental material is a PMMA- and/or MMA-free dental material.

According to a preferred variant of the method, a mixture of monomers and optionally polymers, being present as single phase in the mixture, is used as ready for use, polymerisable dental material, wherein the total composition comprises 100% by weight and the mixture comprises
(1) 25 to 90% by weight, in particular 20 to 40% by weight or 40 to 90% by weight, at least one urethane (meth)acrylate, in particular urethane dimethacrylate, or a mixture comprising at least two urethane (meth)acrylates, in particular 60 to 85% by weight or, in an alternative, as component (i) 40 to 75% by weight and as component (ii) 5 to 30% by weight, in particular 5 to 20% by weight, preferably 10 to 15% by weight,
(2) 0.5 to 40% by weight, in particular 0.5 to 30% by weight, at least one di(meth)acrylate without urethane groups or a mixture comprising at least two di(meth)acrylates, in particular 5 to 20% by weight, preferably 5 to 15% by weight, particularly preferably about 10% by weight, having a range of variation of +/−5% by weight, preferably +/−2% by weight,
(3) 0 to 40% by weight, in particular 5 to 40% by weight, at least one tri-, tetra- or higher functional (meth)acrylate without urethane groups or a mixture comprising at least two of the afore-mentioned (meth)acrylates, in particular 5 to 20% by weight, preferably 5 to 15% by weight, particularly preferably about 10% by weight, preferably having a range of variation of +/−5% by weight, particularly preferably +/−2% by weight,
   optionally, further components such as inorganic fillers, stabilizers, activators, pigments or initiators may be used additionally to the mixture in order to adjust 100% by weight of the dental composition. A dispersion may be formed from the single phase mixture by adding further components.

In this context, according to an alternative, it is preferred for the dental material to be separated as paste A and paste B in one cartridge (double chamber cartridge) or in two cartridges and to be mixed when discharging. Preferably, mixing ensues by a static mixer provided between cartridge and negative mould. Discharging of the dental material may ensue manually or automatically. According to a further preferred alternative, the dental material is separated in a kit in two separated cartridges, in one cartridge as paste A and in a second cartridge as paste B, or in one cartridge (double chamber cartridge) separately as paste A and B.

Furthermore, it is preferred for the cartridge(s) and the inlet opening of the negative mould to be attachable to one another for charging the dental material into the negative mould. This may be achieved by measures known by the person skilled in the art, such as screwing together, bracing or suchlike. Preferably, the connection between cartridge(s) and inlet opening is sealed outwardly in order that no dental material may emerge from the connecting point.

In addition, according to a preferred variant of the method, charging into the negative mould may ensue pneumatically. This may be ensued automatically or manually by means of a applicator gun or a dispenser, respectively. According to a variant, the method is a pneumatic injection method or, alternatively, a manual one. A pneumatic method, in which, for example, a conventional cuvette is used as negative mould, is preferred. Particularly preferably, charging into the negative mould ensues at a pressure of 0.5 to 10 bar, preferably 1 to 10 bar, in particular 1.1 to 5 bar, preferably of 1.1 to 2.5 bar, further preferably about 1.5 to 2.5 or about 2.0 bar with +/−0.5 bar. In this context, the cartridge has a static mixer which is attached in the inlet opening of the negative mould. Polymerisation may preferably ensue at room temperature or at 40 to 70° C., preferably about 55° C. +/−5° C.

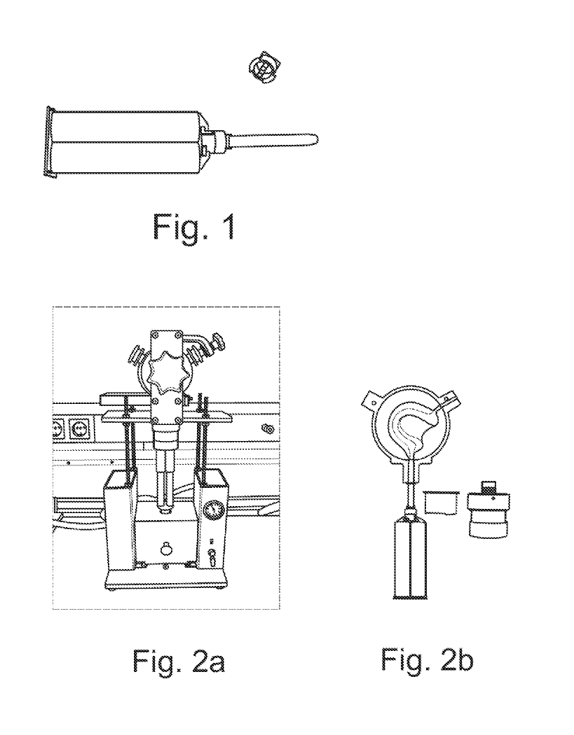

Charging of the dental material preferably also ensues during polymerisation in order to compensate shrinkage potentially arising when polymerising. In this way, exact prosthetic moulded parts, in particular without subsequent marginal gaps, are obtained. Polymerisation may ensue by means of hot-polymerisation or via autopolymerisation. The method according to the invention is illustrated in FIGS. 1 to 4.

According to the method according to the invention, it is preferably polymerised and subsequently the at least one dental prosthetic moulded part is obtained. Preferably, the dental material described below according to the invention or a kit according to the invention is used in the method. Moreover, each polymerisable dental material, in particular in a cartridge, having a certain viscosity is suitable to be used in the method according to the invention.

The dental material according to invention enables implementation of the method according to the invention without waiting times, as otherwise usual, when mixing the known prosthetic materials from the state of the art comprising MMA and PMMA beads. According to the invention, preferably no light-curing polymerisable dental materials are used in the method. Therefore, a method in which the dental material according to the invention or a kit according to the invention is used ist also a subject matter of the invention.

According to the invention, the object is solved by a paste-like or cream-like, polymerisable dental material or prosthetic material, respectively, in which all components are present in processable and temporally stable consistency and no or only low swelling processes or dissolving processes occur. In addition, the consistency of the unpolymerised, ready for use dental material, in particular of pastes A and/or B, is temporally stable.

The dental material according to the invention is immediately ready for use and has a processable consistency. In a particularly preferred embodiment of the invention, the dental material is present as a paste A and a Paste B, which only differ by the initiators or initiator components of an initiator system.

In the case of a cold-polymerisate both components of the dental material preferably have a very similar composition of the dental material and normally differ by the present initiator.

The 2K-prosthetic material according to the invention is preferably filled into a double chamber cartridge (1:1 double chamber cartridge) or a coaxial cartridge (e.g. Peeler of Sulzer Mixpac), mixing the two components and thereby starting the polymerisation ensues by a static mixer at the cartridge outlet when conveying.

Aside the use of the cold-polymerising prosthetic material immediately from the static mixer, e.g. for repairs and relinings, denture saddles or for flowing into a casting cuvette, the dental material according to the invention including packaging materials may also be used for direct injecting into a cuvette, e.g. by means of the Palajet system or a dispensing gun.

In the case of a paste-like hot-polymerisate, the prosthetic material according to the invention may also be filled into a 1K-cartridge, thence directly be injected via a connection pipe into a cuvette. In order to keep the packaging materials as identical as possible, the hot-polymerisate may alternatively be filled into a double chamber cartridge, in this case the chambers of the cartridge comprise qualitatively and quantitatively identical dental materials. Typically, both chambers of the cartridge comprise material of the same colour. Optionally, one chamber may also comprise colourless material and the colouring component is in the other chamber. The final colour occurs by mixing the two components.

The dental material may be present as hot-polymerisate and may be polymerised by heating. Preferably, the hot-polymerisate is polymerised above 70° C., preferably at greater than or equal to 95° C. In the case, where the dental material is present as auto- or cold-polymerisate, it may be polymerised by mixing pastes A and B.

In addition, due to the ready for use formulation of the prosthetic material and the accordingly appropriate packaging material, contact by hand to allergenic MMA is omitted and also dust exposure by PMMA powder as well as exposure by MMA vapours is eliminated.

Subject matter of the invention is a polymerisable, in particular ready for use, dental material, preferably for the production of dental prosthetic moulded parts, which has a viscosity of 1000 mPa·s to 75.000 mPa·s, in particular it has a viscosity of 1000 mPa·s to 50000 mPa·s, preferably of 5000 to 20000 mPa·s., particularly preferably of 10000 to 15000 mPa·s, and comprises a mixture of monomers and optionally polymers, being present as a single phase in said mixture. Optionally the polymerisable dental material may comprise 0.1 to 50% by weight of at least one inorganic filler being dispersed in the phase of the mixture, wherein the total composition of the dental material is 100% by weight. The viscosity is preferably determined at room temperature, about 23° C. and, preferably, a shear rate of 1/10 sec with a usual viscometer.

A single phase within the meaning of the invention shall be understood to be a polymerisable dental material, whose pure mixture of monomers and optionally polymers—without inorganic filler—is present as single liquid phase. According to the invention, the monomers and polymers are present as single liquid phase without inner phase boundaries. In this respect, the monomers and polymers are not present as dispersion. After adding the inorganic fillers a dispersion is formed from the single phase of the mixtures of monomers and polymers.

According to a preferred variant of the invention, the polymerisable dental material comprises a mixture of monomers and optionally polymers, being present as single phase in the mixture, wherein the total composition of the dental material comprises 100% by weight and the mixture comprises
(1) 25 to 90% by weight, in particular 20 to 40% by weight or 40 to 90% by weight, at least one urethane (meth)acrylate, in particular urethane dimethacrylate, or a mixture comprising at least two urethane (meth)acrylates, in particular 60 to 85% by weight or, in an alternative, as component (i) 40 to 75% by weight and as component (ii) 5 to 30% by weight, in particular 5 to 20% by weight, preferably 10 to 15% by weight,
(2) 0.5 to 40% by weight, in particular 0.5 to 30% by weight, at least one di(meth)acrylate without urethane groups or a mixture comprising at least two di(meth)acrylates, in particular 5 to 20% by weight, preferably 5 to 15% by weight, particularly preferably about 10% by weight, having a range of variation of +/−5% by weight, preferably +/−2% by weight,
(3) 0 to 40% by weight, in particular 5 to 40% by weight, at least one tri-, tetra- or higher functional (meth)acrylate without urethane groups or a mixture comprising at least two of the afore-mentioned (meth)acrylates, in particular 5 to 20% by weight, preferably 5 to 15% by weight, particularly preferably about 10% by weight, preferably having a range of variation of +/−5% by weight, particularly preferably +/−2% by weight, optionally, further components such as inorganic fillers, stabilizers, activators, pigments or initiators may be used additionally to the mixture in order to adjust 100% by weight of the dental composition. A dispersion may be formed from the single phase mixture by adding further components.

According to a particularly preferred embodiment of the invention, the dental material comprises as further components
(4) 0.1 to 60% by weight, in particular 0.1 to 30% by weight, preferably 0.1 to 10% by weight or, alternatively, 10 to 50% by weight, inorganic, amorphous filler or a mixture of inorganic fillers, and/or
(5) 0.01 to 5% by weight of an initiator, in particular of a radical initiator, or an initiator system for hot- or cold-polymerisation or cold-curing, respectively, wherein the total composition of the dental material is 100% by weight.

The dental materials according to the invention may comprise, preferably 0.01 to 2% by weight, at least one colour pigment or a mixture of colour pigments in the total composition.

According to a particularly preferred variant, the dental material comprises as the at least one urethane (meth)acrylate (1), in particular urethane dimethacrylate, or a mixture comprising at least two urethane (meth)acrylates, preferably 10 to 75% by weight in the mixture,

(i) at least one urethane di(meth)acrylate, in particular bis(methacryloxy-2-ethoxycarbonylamino)alkylene, bis(methacryloxy-2ethoxylcarbonylamino)alkylene with alkylene of 2 to 15 C-atoms, in particular a C9-alkylene, is particularly preferred and preferably 5 to 30% in the mixture,
(ii) at least one urethane tri(meth)acrylate, in particular aliphatic polyester of an triurethane triacrylate, aliphatic urethane triacrylate.

The at least one urethane (meth)acrylate or a mixture of urethane (meth)acrylates comprises at least one urethane (meth)acrylate, urethane di(meth)acrylate, urethane tri(meth)acrylate or one multifunctional urethane (meth)acrylate, such as urethane dimethacrylate, urethane di(meth)acrylate from the reaction of an α,ω-functionalised alkyldiisocyanate OCN—(CH₂)ₙ—CNO with n=2 to 20, in particular with n=9 with an HO-functional acrylate, in particular hydroxyethylacrylate, in particular an urethane dimethacrylate, preferably an bis(methacryloxy-2-ethoxycarbonylamino)alkylene, aliphatic polyester of a triurethane triacrylate, diurethane acrylate oligomer, alkyl-functional urethane dimethacrylate oligomers, aromatically functionalised urethane dimethacrylate oligomers, aliphatic unsaturated urethane acrylates, bis(methacryloxy-2-ethoxycarbonylamino)-substituted polyether, aromatic urethane diacrylate oligomers, aliphatic urethane diacrylate oligomers, monofunctional urethane acrylates, aliphatic urethane diacrylates, hexafunctional aliphatic urethane resins, aliphatic urethane triacrylate, UDMA, aliphatic urethane acrylate oligomer, unsaturated aliphatic urethane acrylate.

Bis(methacryloxy-2-ethoxycarbonylamino)alkylene, with alkylene of 2 to 15 C-atoms, in particular a C9-alkylene, is particularly preferred.

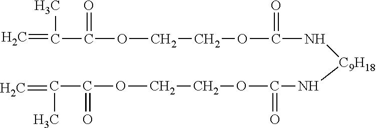

The at least one di(meth)acrylate without urethane groups or mixture of di(meth)acrylate without urethane groups comprising at least two di(meth)acrylates, preferably comprises di-, tri- or tetraethylene glycol di(meth)acrylate, propoxylated neopentylglycol diacrylate, alkyldiol di(meth)acrylate with C2 to C15 in the alkyl group, decanediol di(meth)acrylate, dodecanediol di(meth)acrylate, hexyldecanediol di(meth)acrylate, butanediol di(meth)acrylate, ethylene glycol di(meth)acrylate, polyethylene glycol di(meth)acrylate, ethoxylated/propoxylated bisphenol-A di(meth)acrylate and/or HDDMA (1,6-hexanediol dimethacrylate), bisphenol-A di(meth)acrylate (BPDMA), 1,4-butanediol dimethacrylate (1,4-BDMA), Bis-GMA monomer bisphenol-A glycidylmethacrylate (an addition product of methacrylic acid and bisphenol-A diglycidylether), methacrylate-based difunctional monomer comprising a polyisocyanurate group and/or diethylene glycol di(meth)acrylate. The following difunctional monomers may also be added as diluents (highly fluid acrylates such as triethylene glycol dimethacrylate (TEGDMA) and diethylene glycol dimethacrylate (DEGMA)), for example, for adjusting the viscosity. Decanediol di(meth)acrylate, dodecandiol di(meth)acrylate and/or ethoxylated/propoxylated bisphenol-A di(meth)acrylate are particularly preferred.

The at least one tri-, tetra- or higher function (meth)acrylate without urethane groups or a mixture comprising at least two of the afore-mentioned (meth)acrylates (3) preferably comprises tri- or tetraethylene glycol di(meth)acrylate, trimethylol propane tri(meth)acrylate, methacrylate-based tri- and/or tetra-functional monomer comprising polyisocyanurate groups, tris(2-hydroxyethyl)isocyanurate triacrylate and/or pentaerythritol tetraacrylate.

A preferred mixture comprises derivatives of glycol di(meth)acrylates, such as propoxylated neopentylglycol diacrylate, an urethane diacrylate such as bis(methacryloxy-2-ethoxycarbonylamino)alkylene, with alkylene of 2 to 15 C-atoms, in particular a C9-alkylene, as well as triethylene glycol dimethacrylate (TEGDMA) and diethylene glycol dimethacrylate (DEGMA). These monomers are preferred to adjust viscosity. Decanediol di(meth)acrylate, dodecanediol di(meth)acrylate and/or ethoxylated/propoxylated bisphenol-A di(meth)acrylate are particularly preferred.

Preferably at least one amorphous filler is preferably used as inorganic filler or as a mixture of inorganic fillers in the ready for use dental material according to the invention. Preferably pyrogenic silicic acid or an inorganic filler comprising precipitated silicic acid, dental glasses, such as aluminosilicate glasses or fluoroaluminosilicate glasses, barium aluminium silicate, strontium silicate, strontium borosilicate, lithium silicate, lithium aluminium silicate, phyllosilicates, zeolites, amorphous spherical fillers on oxide or mixed oxide basis, in particular mixed oxides of SiO₂ and ZrO₂, glass fibres and/or carbon fibres, as well as mixtures comprising at least one of the afore mentioned fillers is used, pyrogenic silicic acid and mixed oxides of SiO₂ and ZrO₂ are preferred.

Preferably, the polymerisable dental material according to the invention does not comprises a content of organic polymers, such as polymethacrylic acid methylester (PMMA), polymethylacrylic acid ethylester (PMEA), methacrylic acid methylester (MMA) and/or ethylacrylic acid ester (EMA) as well as cross-linked polymers and/or Co-polymers. Appropriately, the content of organic polymers may have 0.0% by weight, in particular 0.001% by weight to 5.0% by weight, in the total composition. The content of methylmethacrylate (MMA) and/or ethylmethacrylate (EMA) preferably is also about 0.0% by weight. Appropriately, 0.0001 to 0.1% by weight MMA and/or EMEA may be contained, wherein the polymerisable dental material preferably is completely free of MMA and/or EMA.

A further subject matter of the invention is a kit comprising a polymerisable dental material for the production of dental prosthetic moulded parts, wherein the dental material is present as paste having a viscosity of 1000 mPa·s to 75000 mPa·s, in particular of 1000 mPa·s to 50000 mPa·s, preferably having a viscosity of 5000 to 20000 mPa·s., preferably of 5000 to 15000 mPa·s, in a cartridge.

According to the invention, it is particularly preferred for the monomeric and optionally polymeric components to be present in the polymerisable dental material in one phase, in particular as liquid phase, preferably as single phase (homogenous phase), the phase is preferably present as single liquid phase, in which inorganic fillers are optionally dispersed. The presence of a phase may be observed microscopically. According to the invention, a paste-like, immediately ready for use dental material is present as one-component or two-component dental material. The paste-like dental material is present as ready for use dispersion in the case of adding inorganic fillers and as ready for use single-phase phase without adding these fillers.

Furthermore, a subject matter of the invention is a kit comprising the ready for use, polymerisable dental material in at least one cartridge or in two cartridges, in particular the cartridge is a coaxial cartridge or a double chamber cartridge. Furthermore, it is preferred for the cartridge to be formed such that it is appropriate for the use in an injection method for the production of dental prosthetic moulded parts. Preferably, the dental material is present as ready for use and storable paste having a viscosity of 1000 mPa·s to 75000 mPa·s. Particularly preferably, the viscosity is in the range of 1000 mPa·s to 50000mPa·s, preferably 1000 to 20000 mPa·s., preferably of 5000 to 15000 mPa·s.

In addition, a subject matter of the invention is a kit comprising a paste A and a paste B, wherein pastes A and B, each independently, comprise a mixture of monomers and optionally polymers, each being present as a single phase, in particular as liquid phase. In this context, in particular, it is further preferred for the (a) paste A to have a viscosity of 1000 mPa·s to 75000 mPa·s, in particular a viscosity of 1000 mPa·s to 50000 mPa·s, preferably 1000 to 15000 mPa·s., preferably of 2000 to 10000 mPa·s and to comprise (a1) at least one urethane (meth)acrylate, (a2) at least one di(meth)acrylate without urethane groups, (a3) at least one tri-, tetra- or higher functional (meth)acrylate without urethane groups, (a4) optionally, at least one inorganic filler, and wherein the (b) paste B has a viscosity of 1000 mPa·s to 75000 mPa·s, in particular a viscosity of 1000 mPa·s to 50000 mPa·s, preferably 5000 to 20000 mPa·s., preferably of 5000 to 17500 mPa·s, and comprises (b1) at least one urethane (meth)acrylate,

(b2) at least one di(meth)acrylate without urethane groups, (b3) at least one tri-, tetra- or higher functional (meth)acrylate without urethane groups, (b4) optionally, at least one inorganic filler, wherein, optionally, the filler (a4) and/or (b4) is dispersed in the phase of the mixture,
and wherein at least one of the pastes A and/or B comprises as component (a5) or (b5), respectively, at least one content of a (a5) radical initiator or radical initiator system for hot- or cold-polymerisation, and wherein the total composition is 100% by weight.

Furthermore, a subject matter of the invention is a kit comprising pastes A and B, wherein the pastes may be present in the following alternatives: (i) wherein at least one of the pastes A and/or B comprises as component (a5) or (b5), respectively, at least one content of a radical initiator comprising a peroxide compound and optionally at least one amine, or (ii) wherein (a5) and (b5) are separated into the pastes A and B and comprise at least a part of a redox system in each case as components (a5) or (b5), respectively, only when mixing pastes A and B. For example, a peroxide and an amine are separated by one another, each in one of the pastes A and B. Likewise, (i) at least one of the pastes A and/or B may comprise an azo compound as component (a5) or (b5), respectively, at least a content of a radical initiator, the compound being separated into the pastes A and B.

According to the invention, pastes A and B only differ by the initiator complex.

In addition, it may be preferred for paste A to comprise at least a content of a radical initiator comprising a peroxide compound and, optionally, at least one amine, and for paste B to comprise an activator, such as an aromatic amine derivative. Preferred compounds are exemplary listed in the following. Likewise preferably, one of the pastes A or B may optionally comprise as component (a5) or (b5), respectively, at least one copper compound, at least one halide source and at least one barbituric acid(s) or barbituric acid derivatives. Preferred components (a5) and (b5) are listed in the following. The person skilled in the art knows, that further usual initiator/-systems may also be used in the ready for use, polymerisable dental composition.

A further subject matter of the invention is a kit comprising a ready for use, polymerisable dental composition, comprising a (a) paste A having a viscosity of 1000 mPa·s to 75000 mPa·s, in particular of 1000 mPa·s to 50000 mPa·s, preferably having a viscosity of 1000 to 15000 mPa·s., preferably of 2000 to 10000 mPa·s, wherein the past has a mixture comprising

(a1) 25 to 80% by weight, in particular 20 to 40% by weight or 40 to 80% by weight, at least one urethane (meth)acrylate, in particular urethane dimethacrylate or a mixture comprising at least two urethane (meth)acrylates,
(a2) 0.5 to 40% by weight, in particular 0.5 to 30% by weight, at least one di(meth)acrylate without urethane groups or a mixture comprising at least two di(meth)acrylates,
(a3) 0 to 40% by weight, in particular 5 to 40% by weight, at least one tri-, tetra- or higher functional (meth)acrylate without urethane groups or a mixture comprising at least two of the afore mentioned (meth)acrylates,
(a4) optionally, 0.1 to 60% by weight, in particular 0.1 to 45% by weight, preferably 0.1 to 10% by weight, inorganic filler, in particular at least one amorphous inorganic filler, as well as a
(b) paste B having a viscosity of 1000 mPa·s to 75000 mPa·s, in particular of 1000 mPa·s to 50000 mPa·s, preferably having a viscosity of 5000 to 20000 mPa·s., preferably of 5000 to 17500 mPa·s, wherein paste B has a mixture comprising
(b1) 25 to 80% by weight, in particular 20 to 40% by weight or 40 to 80% by weight, at least one urethane (meth)acrylate, in particular urethane dimethacrylate, or a mixture comprising at least two urethane (meth)acrylates,
(b2) 0.5 to 40% by weight, in particular 0.5 to 30% by weight, at least one di(meth)acrylate without urethane groups or a mixture comprising at least two di(meth)acrylates,
(b3) 0 to 40% by weight, in particular 5 to 40% by weight, at least one tri-, tetra- or higher functional (meth)acrylate without urethane groups or a mixture comprising at least two of the afore mentioned (meth)acrylates,
(b4) optionally, 0.1 to 60% by weight, in particular 0.1 to 45% by weight, preferably 0.1 to 10% by weight, inorganic filler, preferably at least one amorphous, inorganic filler,
and wherein, optionally, at least one of the pastes A and/or B or both pastes A and B comprises as component (a5) or (b5), respectively, at least 0.01 to 5% by weight of a (a5) radical initiator or radical initiator system for hot- or cold-polymerisation, wherein the total composition of the respective paste A and B is 100% by weight.

According to a preferred embodiment, paste A comprises as (a4) a peroxide (oxidant), in particular cumene hydroperoxide, and paste B comprises as (b4) at least one compound having an ionic halogen atom and at least one copper salt or copper complex, in particular copper (II) chloride, at least one barbituric acid or thiobarbituric acid, or, in particular an acetylthiourea, and/or at least one chloride source, such as methyltrioctylammonium chloride, and, optionally, at least one polymerisation regulator, such as Y-terpinene. The person skilled in the art knows that component (b4) may also be used as (a4) and (a4) as (b4).

According to the invention, it is preferred for pastes A and B to be respectively separated in a cartridge and preferably be mixed about 1 to 1. Furthermore, it is preferred for pastes A and B to have an essentially qualitatively and quantitatively identical composition regarding components (a1) to (a4) and (b1) to (b4), in particular the maximum quantitative deviation is 10% by weight of the respective compound. Optionally, the ready for use dental material may also be present in a cartridge having one chamber.

Usual pigments may be added to the dental material for adjusting the aesthetical appearance.

A particularly preferred kit according to the invention comprises a cartridge having an electric sensor. The sensor serves for identification of the cartridge in a conveying system, such as, for example, the Palajet system, in which the cartridge is insertable. The sensor may also be developed to be identified in another conveying system.

In addition, a subject matter of the invention is a ready for use, polymerisable dental material which preferably comprises in the dental material or in one of pastes (A) or (B) or in (A) and (B) additionally at least one or more substance(s) from the group of pigments, stabilizers, regulators, antimicrobial additives, UV-absorbers, thixotroping agents. Such additives—like pigments, stabilizers and regulators—are used in rather small amounts, e.g. in total 0.01 to 3.0% by weight, particularly preferably the dental material comprises 0.01 to 1.0% by weight of the afore-mentioned substances, based on the total mass of the dental material. Appropriate stabilizers include, for example hydroquinone monomethylether or 2,6-di-tert.-butyl-4-methylphenol (BHT).

The following initiators and/or initiator systems for auto- or cold polymerisation may comprise a) at least one initiator, in particular at least one peroxide, in particular LPO: dilauroylperoxide, BPO: dibenzoylperoxide, t-BPEH: tert.-butylper-2-ethylhexanoate, DTBP: di-tert.-butylperoxide, and b) at least one activator, in particular at least one aromatic amine, such as N,N-dimethyl-p-toluidine, N,N-dihydroxyethyl-p-toluidine and/or p-dibenzylamino benzoic acid diethylester, or c) at least one initiator system selected from redox systems, in particular a combination selected from dibenzoylperoxide and dilauroylperoxide with amines selected form N,N-dimethyl-p-toluidine, N,N-dihydroxyethyl-p-toluidine and p-dimethylamino benzoic acid diethylester, or a redox system comprising a peroxide, an a reductant selected form ascorbic acid, ascorbic acid derivative, barbituric acid or a barbituric acid derivative, sulfinic acid, sulfinic acid derivative, a redox system comprising

(i) barbituric acid or thiobarbituric acid or a barbituric acid derivative or thiobarbituric acid derivative and (ii) at least one copper salt or copper complex and
(iii) at least one compound having an ionic halogen atom is particularly preferred, a redox system comprising 1-benzyl-5-phenylbarbituric acid, copper acetylacetonate and benzyldibutylammonium chloride is particularly preferred. Particularly preferably, the polymerisation in 2-component dental material is started by a barbituric acid derivative. In an alternative, the dental material preferably is free of peroxides, whilst in another alternative, as repairing material, production of relining of prostheses and extension of prostheses, it comprises peroxides for faster curing. Component (i) may be present in paste A and components (ii) and (iii) may be present in paste B or vice versa.

Azo compounds, such as AIBN (2′,2′-azobis(isobutyrobitrile)), may be used for hot-polymerisation and/or camphorquinone optionally with amines selected from N,N-dimethyl-p-toluidine, N,N-dihydroxyethyl-p-toluidine and p-dimethylamino benzoic acid diethylester may be used for radiation curing, or also with further photoinitiators for dual curing.

According to a preferred variant of the method, paste A and paste B, in particular pastes A and B of the kit according to the invention comprising a cartridge, are mixed in weight ratio of 1:50 to 50:1, in particular in weight ratio 1:2 to 2:1, particularly preferably of about 1:1, in particular with a range of variation of plus/minus 0.5, preferably plus/minus 0.25.

According to the invention, mixing pastes A and B may be ensued by means of a static mixer at the outlet opening of a double chamber cartridge. Likewise, mixing may be ensued by usual mixers, for example a static mixer or a dynamic mixer.

A pigmented, completely polymerised dental material is also a subject matter of the invention. In addition, an unpigmented, completely polymerised dental material is a subject matter of the invention.

A further subject matter of the invention is a method for the production of dental prosthetic moulded parts, in which from a kit according to the invention comprising the ready for use, polymerisable dental material, optionally as paste A and B, in at least one cartridge, two cartridges or several cartridges, the dental material is discharged optionally by mixing and charged into a negative mould for the production of at least one dental prosthetic moulded part, and, optionally, directly polymerised. Preferably, disposable cartridges are used.

In this context, several cartridges may be used, for example, in order to obtain defined colours/layers via adding the pastes by means of different colourfully adjusted pastes and preferably by means of previously defined mixing ratios of the pastes. Dental bridges, parts of bridges, prosthesis base parts, teeth, veneers etc. may be produced with the method according to the invention.

In addition, a subject matter of the invention is a method for the production of dental prosthetic moulded parts, in which from the kit the ready for use, polymerisable dental material as paste A and B in two separated cartridges, is discharged optionally by mixing and charged into a negative mould for the production of at least one dental prosthetic moulded part and, optionally, directly, in particular within 2 to 3 minutes, the polymerisation is started. Preferably, the polymerisable dental material is homogenously mixed in a static mixer on the cartridge, in particular homogenous mixing ensues during injection into a negative mould.

Furthermore, it is preferred for the dental material or pastes A and B to be mixed by means of pneumatically or electrically or magnetically driven injection devices and to be directly injected into a negative mould, in particular a cuvette. Further advantages of the dental material according to the invention are that the ready for use dental material may be mixed by means of a hand-operated discharging device and be directly injected into a negative mould, in particular in a cuvette. Alternatively, the ready for use dental material may be present in a cartridge or double chamber cartridge comprising an electronic component which is identified by a mixing system, for example the Palajet system, and enables processing of the material in the mixing system, such as Palajet.

A further subject matter of the invention is the use of a ready for use dental material according to the invention or of a kit according to the invention for the production of dental prosthetic moulded parts, parts of dental prostheses, such as dental partial prosthesis, dental total prostheses, orthopedic prostheses or parts thereof, artificial teeth, veneers, inlays, onlays, superstructures, dental carrier structures, bridges, crowns, relinings, denture saddles, bone prostheses, joint prostheses, revision total joint endoprostheses and/or spacers, in particular in a manual or pneumatic injection method, preferably in a pneumatic method under a pressure of 0.5 to 10 bar, preferably at 1 to 5 bar, further preferably at 1.5 to 5 bar or at 2 to 5 bar.

Preferably, the use ensues by charging the dental composition, after mixing or when discharging and optionally mixing, from the cartridge directly in a cuvette or negative mould for the production of at least one dental prosthetic moulded part and polymerising.

A particular advantage of the ready for use, polymerisable dental materials according to the invention is that no swelling time is necessary anymore, unlike usual dental materials, in particular dental prosthesis materials.

Advantageously, the amount of the dental material or of pastes A and B in the cartridges respectively corresponds to the amount required for the production of a partial or total prosthesis. Usually, 20 to 30 g are required for the production of a total prosthesis.

### EXAMPLES

TABLE 1
Formula cold-polymerising prosthetic material
according to the invention (stated in % by weight)

| | Component A | Component B |
|---|---|---|
| urethane dimethacrylate | 62% | 62% |
| aliphatic polyester triurethane acrylate | 13% | 13% |
| dodecandiol dimethacrylate | 5% | 5% |
| ethoxylated bisphenol-A dimethacrylate | 5% | 5% |
| alkoxylated pentaerythritol tetraacrylate | 10% | 10% |
| pyrogenic silicic acid | 4% | 4% |
| BHT | <0.5% | <0.5% |
| Cu(II)Cl₂ | 0.001% | X |
| methyltrioctylammonium chloride | 0.2% | X |
| PBS (phenylbenzylbarbituric acid) | X | 1% |
| BBS (butylbarbituric acid) | X | 0.2% |

A swelling time of the mixture is not necessary anymore. The dental mixture is immediately ready for use.

TABLE 2
Comparison of processing times

| | According to the | PalaXpress (not |
|---|---|---|
| | invention | according to the invention) |
| weighing | 0 | 30 sec. |
| mixing | 0 | 30 sec. |
| swelling | 0 | 3 min. |
| injecting | 10 min. | 10 min. |
| polymerising | 30 min. | 30 min. |

TABLE 3
Comparison of properties of the polymerisable dental material according to the
invention when processing in mixing unit and in the static mixer of a double chamber
cartridge (stated in % by weight)

| | | | Example 2 | |
|---|---|---|---|---|
| | | | (Composition | |
| | Example 1 | | as Example 1) | |
| | Component | Component | Component | Component |
| | A | B | A | B |
| Mixing ratio | 1:1 | | 1:1 | |
| propoxylated | 20.000 | 20.000 | 20.000 | 20.000 |
| neopentylglycol diacrylate | | | | |
| UDMA | 30.000 | 30.000 | 30.000 | 30.000 |
| TEDMA | 9.000 | 9.000 | 9.000 | 9.000 |
| nano-part SiO₂ | 40.000 | 39.500 | 40.000 | 39.500 |
| Cu(II)Cl₂ | | 0.100 | | 0.100 |
| methyltrioctylammonium chloride | | 0.170 | | 0.170 |
| Y-terpinene | | 0.230 | | 0.230 |
| cumene hydroperoxide | 1.000 | | 1.000 | |
| acetylthiourea | | 1.000 | | 1.000 |
| total | 100.00 | 100.00 | 100.00 | 100.00 |

| | |
|---|---|
| Mixing the pastes | Mixing the pastes |

| | | | |
|---|---|---|---|
| Paste A | Paste B | Paste A | Paste B |

| | | | |
|---|---|---|---|
| flexural | >60 [MPa] | 64.9 | 66.9 |
| strength | | | |
| E-modulus | >1500 [MPa] | 2463 | 2240 |
| curing | processing via | x | |
| | Hausschild^{[1]} | | |
| | processing by | | x |
| | static mixer^{[2]} | | |
| | pressure unit | x | x |
| | Palamat elite | | |
| | 55° C. 20 min | | |

^{[1]}Mixing components A and B according to the principle of rotary mixer in the mixing unit (Speedmixer 150);
^{[2]}Mixing by stating mixer of the double cartridge

Ready for use pastes A have a viscosity of about 7600 mPa·s (determined at a shear rate of 1/10 sec), pastes B have a viscosity of about 14100 mPa·s under the terms mentioned. A 1:1 mixture of the two pastes A and B has a viscosity of about 12900 mPa·s. The polymerisable dental material meets the requirements of DIN ISO 20795-1: 2013-06, if the prosthesis material is mixed according to both methods. Thus, a ready for use dental material for the production of prostheses may be provided in a simple and economic manner via the method according to the invention, in particular with a polymerisable, ready for use dental material according to the invention having a viscosity of 2000 mPa·s to 15000 mPa·s. As described above, the method according to the invention reduces the number of steps and the time spend in the production of prostheses.

Packaging materials+mixer: The two components A and B are filled in a customary double chamber cartridge, mixing in ratio 1:1. Those cartridges are being offered, for example, by the companies Sulzer Mixpac or Ritter. The static mixer shall be as short as possible having an outer diameter of approx. 8 mm.

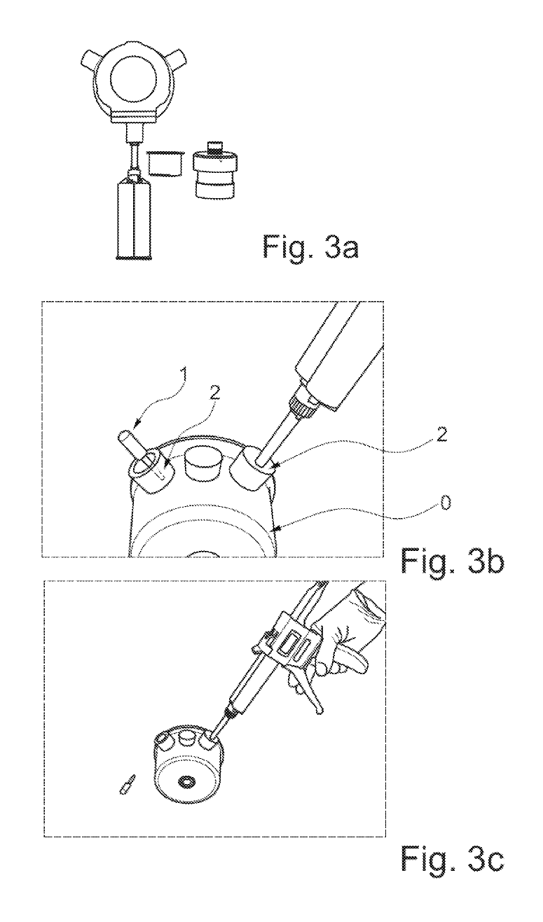

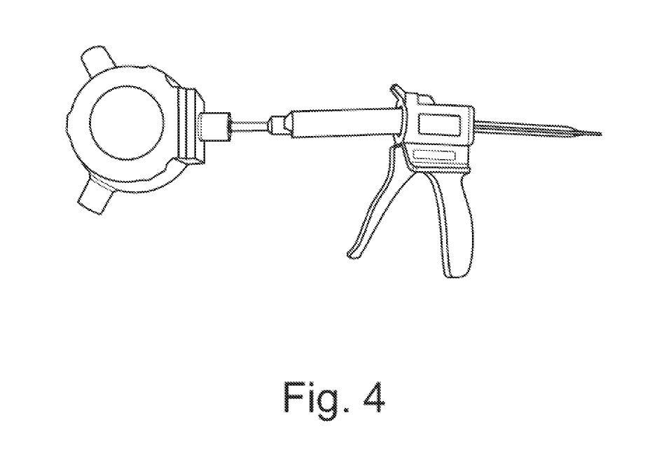

The use according to the invention is shown in the figures. According to FIG. 1, a double chamber cartridge is filled with the polymerisable dental material according to the invention. FIGS. 3a, 3b and 3c show the use in a hand injection method for the production of prosthetic moulded parts, in which the dental material may directly be filled by the static mixer into the casting cuvette in order to polymerise in the cuvette (FIG. 3b: casting cuvette, FIG. 4: Palajet cuvette).

Pneumatic injection method (Palajet): The static mixer screwed on the double chamber cartridge is inserted into the injection opening of a Palajet cuvette, as shown in FIGS. 2a and 2b. After fixing the cuvette in the Palajet and sealing of the injection opening, the dental material is injected from the cartridge into the cuvette. During polymerisation of the dental material in the cuvette, further material is conveyed from the cartridge in order to compensate polymerisation shrinkage. In this way, exact prostheses result. According to the immediate usability shown in the figures of the ready for use, polymerisable dental material, time for the production of the prostheses may considerably be reduced for the user. In addition, direct contact with the skin or breathing of volatile components is being avoided using the dental material according to the invention.

1. A method for the production of dental prosthetic moulded parts, comprising the steps of
using a ready for use, polymerisable dental material as at least one paste in at least one cartridge,
discharging the ready for use dental material, optionally by mixing the dental material, and
charging the dental material into a negative mould for the production of at least one dental prosthetic moulded part, and
optionally, polymerising the dental material.

2. The method according to claim 1, wherein the dental material is separated as paste A and paste B in one cartridge or in two cartridges and is mixed when discharging.

3. The method according to claim 1, wherein charging into the negative mould ensues pneumatically.

4. The method according to claim 1, wherein during polymerisation further dental material is charged into the negative mould.

5. A polymerisable dental material for the production of dental prosthetic moulded parts having a viscosity of 1000 mPa·s to 75000 mPa·s and comprising a mixture of monomers and optionally polymers, being present as single phase in the mixture.

6. The dental material according to claim 5, wherein the mixture of monomers and optionally polymers, being present as single phase, comprises p1 (1) 25 to 90% by weight at least one urethane (meth)acrylate, or a mixture comprising at least two urethane (meth)acrylates,
(2) 0.5 to 40% by weight at least one di(meth)acrylate without urethane groups or a mixture comprising at least two di(meth)acrylates,
(3) 0 to 40% by weight at least one tri-, tetra- or higher functional (meth)acrylate without urethane groups or a mixture comprising at least two of the afore mentioned (meth)acrylates,
based on the total composition of the dental material of 100% by weight.

7. The dental material according to claim 5, wherein 0.1 to 60% by weight of at least one inorganic filler is dispersed in the phase, wherein the total composition of the dental material is 100% by weight.

8. The dental material according to claim 5 comprising
(4) 0.1 to 60% by weight inorganic, amorphous filler or a mixture of inorganic fillers, and/or
(5) 0.01 to 5% by weight of an initiator or initiator system for hot- or cold-polymerisation, wherein the total composition of the dental material is 100% by weight.

9. The dental material according to claim 5 comprising (i) at least one urethane di(meth)acrylate, and (ii) at least one urethane tri(meth)acrylate.

10. The dental material according to claim 5, wherein at least one urethane (meth)acrylate, urethane di(meth)acrylate, urethane tri(meth)acrylate or one multifunctional urethane (meth)acrylate is contained, such as comprising urethane dimethacrylate, urethane di(meth)acrylate from the reaction of an α,ω-functionalised alkyldiisocyanate OCN—(CH₂)ₙ—CNO with n=2 to 20, with an HO functional acrylate, an urethane dimethacrylate, an bis(methacryloxy-2-ethoxycarbonylamino)alkylene aliphatic polyester of a triurethane triacrylate, diurethane acrylate oligomer, alkyl-functional urethane dimethacrylate oligomers, aromatically functionalised urethane dimethacrylate oligomers, aliphatic unsaturated urethane acrylates, bis(methacryloxy-2-ethoxycarbonylamino)-substituted polyether, aromatic urethane diacrylate oligomers, aliphatic urethane diacrylate oligomers, monofunctional urethane acrylates, aliphatic urethane diacrylates, hexafunctional aliphatic urethane resins, aliphatic urethane triacrylate, UDMA, aliphatic urethane acrylate oligomer, unsaturated aliphatic urethane acrylate.

11. The dental material according to claim 5, wherein at least one di(meth)acrylate without urethane groups comprising
di-, tri- or tetraethylene glycol di(meth)acrylate, propoxylated neopentylglycol diacrylate, alkyldiol di(meth)acrylate with C2 to C15 in the alkyl group, decanediol di(meth)acrylate, dodecanediol di(meth)acrylate, hexyldecanediol di(meth)acrylate, butanediol di(meth)acrylate, ethylene glycol di(meth)acrylate, polyethylene glycol di(meth)acrylate, ethoxylated/propoxylated bisphenol-A di(meth)acrylate and/or HDDMA (1,6-hexanediol dimethacrylate), bisphenol-A di(meth)acrylate (BPDMA), 1,4-butanediol dimethacrylate (1,4-BDMA), Bis-GMA monomer bisphenol-A glycidylmethacrylate (an addition product of methacrylic acid and bisphenol-A diglycidylether), methacrylate-based difunctional monomer comprising a polyisocyanurate group and/or diethylene glycol di(meth)acrylate is contained.

12. The dental material according to claim 5, wherein at least one tri-, tetra- or higher function (meth)acrylate without urethane groups comprises tri- or tetraethylene glycol di(meth)acrylate, trimethylol propane tri(meth)acrylate, methacrylate-based tri- and/or tetra-functional monomer comprising polyisocyanurate groups, tris(2-hydroxyethyl)isocyanurate triacrylate and/or pentaerythritol tetraacrylate.

13. The dental material according to claim 5, wherein the inorganic filler is an amorphous inorganic filler, comprising pyrogenic silicic acid or an inorganic filler comprising precipitated silicic acid, dental glasses, such as aluminosilicate glasses or fluoroaluminosilicate glasses, barium aluminium silicate, strontium silicate, strontium borosilicate, lithium silicate, lithium aluminium silicate, phyllosilicates, zeolites, amorphous spherical fillers on oxide or mixed oxide basis, glass fibres and/or carbon fibres, as well as mixtures comprising at least one of the afore-mentioned fillers.

14. A kit comprising a polymerisable dental material for the production of dental prosthetic moulded parts, wherein the dental material is ready for use as paste having a viscosity of 1000 mPa·s to 75000 mPa·s in at least one cartridge.

15. The kit having a paste A and a paste B, wherein pastes A and B, each independently, comprise a mixture of monomers and optionally polymers, each being present as a single phase in the mixture and, in particular, wherein
(a) paste A has a viscosity of 1000 mPa·s to 75000 mPa·s, comprises
(a1) at least one urethane (meth)acrylate,
(a2) at least one di(meth)acrylate without urethane groups,
(a3) at least one tri-, tetra- or higher functional (meth)acrylate without urethane groups, and
(a4) optionally, at least one inorganic filler, and wherein
(b) paste B has a viscosity of 1000 mPa·s to 75000 mPa·s, and comprises
(b1) at least one urethane (meth)acrylate,
(b2) at least one di(meth)acrylate without urethane groups,
(b3) at least one tri-, tetra- or higher functional (meth)acrylate without urethane groups, and
(b4) optionally, at least one inorganic filler,
wherein the optional filler (a4) and/or (b4) is dispersed in the phase of the mixture, and wherein at least one of the pastes A and/or B comprises as component (a5) or (b5), respectively, at least one content of a (a5) radical initiator or radical initiator system for hot- or cold-polymerisation, wherein the total composition in paste A and B is 100% by weight in each case.

16. The kit according to claim 15, wherein
(a) paste A has a viscosity of 1000 mPa·s to 75000 mPa·s, and comprises a mixture of
(a1) 25 to 80% by weight at least one urethane (meth)acrylate, or a mixture comprising at least two urethane (meth)acrylates,
(a2) 0.5 to 40% by weight at least one di(meth)acrylate without urethane groups or a mixture comprising at least two di(meth)acrylates,
(a3) 0 to 40% by weight at least one tri-, tetra- or higher functional (meth)acrylate without urethane groups or a mixture comprising at least two of the afore mentioned (meth)acrylates,
(a4) optionally, 0.1 to 60% by weight inorganic filler and
(b) paste B has a viscosity of 1000 mPa·s to 75000 mPa·s, and comprises a mixture of
(b1) 25 to 80% by weight at least one urethane (meth)acrylate, or a mixture comprising at least two urethane (meth)acrylates,
(b2) 0.5 to 40% by weight at least one di(meth)acrylate without urethane groups or a mixture comprising at least two di(meth)acrylates,
(b3) 0 to 40% by weight at least one tri-, tetra- or higher functional (meth)acrylate without urethane groups or a mixture comprising at least two of the afore mentioned (meth)acrylates,
(b4) optionally, 0.1 to 60% by weight inorganic filler, and wherein at least one of the pastes A and/or B or both pastes A and B comprises as component (a5) or (b5), respectively, at least 0.01 to 5% by weight of a (a5) radical initiator or radical initiator system for hot- or cold-polymerisation, wherein the total composition in paste A and B is 100% by weight in each case.

17. The kit according to claim 14, wherein pastes A and B are respectively separated in a cartridge.

18. The kit according to claim 17, wherein at least one cartridge has an electric sensor.

19. A dental material according to claim 5, being completely polymerised.

20. Method of using a kit according to claim 14 comprising the polymerisable dental material for the production of dental prosthetic moulded parts.

21. Method of using a dental material according to claim 5 for the production of dental prosthetic moulded parts, parts of dental prostheses, dental total prostheses, orthopedic prostheses or parts thereof, artificial teeth, veneers, inlays, onlays, superstructures, dental carrier structures, bridges, crowns, relinings, denture saddles, bone prostheses, joint prostheses, revision total joint endoprostheses and/or spacers.

22. The method according to claim 21, wherein the dental material is, after mixing or when discharging and optionally mixing, directly charged from the cartridge into a negative mould or cuvette for the production of at least one dental prosthetic moulded part and polymerised.
